(19) United States
(12) Patent Application Publication
(10) Pub. No.: US 2007/0196293 A1
(43) Pub. Date: Aug. 23, 2007

(54) Compositions and methods for treating photo damaged skin

(76) Inventors: Jie Zhang, Salt Lake City, UT (US); Kevin S. Warner, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/640,102

(22) Filed: Dec. 14, 2006

Related U.S. Application Data
(63) Continuation-in-part of application No. 11/146,917, filed on Jun. 6, 2005.
(60) Provisional application No. 60/750,637, filed on Dec. 14, 2005.
(60) Provisional application No. 60/577,536, filed on Jun. 7, 2004.

(51) Int. Cl. A61K 31/4745; A61K 35/64

(52) U.S. Cl. 424/59; 514/292; 424/539

(57) ABSTRACT

The present invention is drawn to formulations, methods, and solidified layers for topical delivery of an immune modulating agent for treatment of photo damaged skin. The formulation can include an immune modulating agent, a solvent vehicle, and a solidifying agent. The solvent vehicle can include a volatile solvent system having one or more volatile solvent, and a non-volatile solvent system having one or more non-volatile solvent, wherein non-volatile solvent system is capable of facilitating the delivery of immune modulating agent at therapeutically effective rates over a sustained period of time. The formulation can have a viscosity suitable for application to a skin surface prior to evaporation of the volatile solvents system. When applied to the skin, the formulation can form a solidified layer after at least a portion of the volatile solvent system is evaporated.

This application claims the benefit of U.S. Provisional Application No. 60/750,637 filed on Dec. 14, 2005, and is a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to formulations, methods, and solidified layers developed for cosmetic treatment of skin, especially photo damaged skin. More particularly, the present invention relates to adhesive solidifying formulations having a viscosity suitable for application to a skin surface as a layer, and which form a sustained drug-delivering adhesive solidified layer on the skin.

### BACKGROUND OF THE INVENTION

It is believed that topical application of immune activators such as imiquimod can be used to treat photo damages and premature aging of the skin, which are characterized by fine lines, wrinkles, roughness, dryness, laxity, and/or irregular pigmentation. For example, treatment of visible signs of photoaging with an imiquimod over several weeks can improve the morphology and appearance of photodamaged skin. However, the only commercially available dosage form of imiquimod, Aldara Cream from 3M, was not designed or approved for treating photodamaged skin, i.e. it was approved for treating genital warts and basal cell carcinoma. After the cream is applied on and rubbed “into” the skin, most of the drug does not really get into the skin. Instead, most of the drug stays on the surface of the skin for a long duration during which it is subject to unintentional removal. For example, the cream applied on a subject's face and forehead before bedtime can be removed by the pillow or blanket during the night.

Further, in general, while patches and semisolid formulations are widely used to deliver drugs topically, they both have significant limitations. For example, most semisolid formulations usually contain solvent(s), such as water and ethanol, which are volatile and thus evaporate shortly after application. The evaporation of such solvents can cause a significant decrease or even termination of dermal drug delivery, which may not be desirable in many cases. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional semisolid formulations applied to the skin may not contain sufficient quantity of active drug to achieve sustained delivery over long periods of time. Additionally, traditional semisolid formulations are often subject to unintentional removal due to contact with objects such as clothing, which may compromise the sustained delivery and/or undesirably soil clothing. Drugs present in a semisolid formulation may also be unintentionally delivered to persons who come in contact with a subject undergoing treatment with a topical semisolid formulation. Additionally, patches are not ideal for treating skin on the face for many obvious reasons.

In view of the shortcomings of many of the current delivery systems, it would be desirable to provide systems, formulations, and/or methods for cosmetically treating skin, especially photo damaged skin, that can i) provide sustained drug delivery over long periods of time; ii) are not vulnerable to unintentional removal by contact with clothing, other objects, or people for the duration of the application time; iii) can be applied to a facial skin area with ease and without causing discomfort or poor contact to skin; and/or iv) can be easily removed after application and use.

### SUMMARY OF THE INVENTION

Thus, it would be advantageous to develop formulations for delivering immune activating agents, systems, and/or methods for delivering the same. Such formulations can include adhesive solidifying compositions or formulations having a viscosity suitable for application to the skin surface to be treated as a layer and which form a solidified layer on the skin that is removable after use. In accordance with this, a formulation for treating photo damaged human skin, comprising an immune modulating agent, a solvent vehicle, and a solidifying agent. The solvent vehicle can comprise a volatile solvent system including at least one volatile solvent, and a non-volatile solvent system including at least one non-volatile solvent. The formulation can have a viscosity suitable for application and adhesion to a skin surface prior to evaporation of the volatile solvent system, and can be applied as a layer to the skin surface to form a solidified layer after at least partial evaporation of the volatile solvent system. The drug can continue to be topically delivered after the volatile solvent system is substantially evaporated.

In another embodiment, a method of treating photo damaged skin can comprise applying a layer of a formulation to an area of skin exhibiting aging, wrinkles, or photo damage. The formulation can comprise an immune modulating agent, a solvent vehicle, and a solidifying agent. The solvent vehicle can include a volatile solvent system including at least one volatile solvent, and a non-volatile solvent system including at least one non-volatile solvent, wherein the non-volatile solvent system is capable of facilitating topical delivery of the immune modulating agent at a therapeutically effective rate to the skin over a sustained period of time. The formulation can have a viscosity suitable for application and adhesion to the skin surface prior to evaporation of the volatile solvent system. Other steps include solidifying the formulation on the skin to form a solidified layer by at least partial evaporation of the volatile solvent system, and dermally delivering the immune modulating agent from the solidified layer to the area of skin over a sustained period of time at a therapeutically effective rate for treating photo damaged human skin.

In another embodiment, an adhesive solidifying formulation for treating photo damaged human skin can comprise an immune modulating agent, and at least one member selected from the group consisting of isostearic acid, triacetin, sorbitan monolaurate, and combinations thereof.

In another embodiment, a solidified layer for treating photo damaged human skin can comprise an immune modulating agent, a non-volatile solvent system, and a solidifying agent. The non-volatile solvent system can comprise at least one non-volatile solvent, and the system can be capable of facilitating the delivery of the immune modulating agent at a therapeutically effective rate over a sustained period of time. The solidified layer can be capable of adhering to a human skin surface for at least two hours.

Additional features and advantages of the invention will be apparent from the following detailed description which illustrate, by way of example, features of the invention.

### DETAILED DESCRIPTION OF THE PREFERRED EMBODIMENTS

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken), finger and toe nail surfaces, and mucosal surfaces that are usually at least partially exposed to air such as lips, genital and anal mucosa, and nasal and oral mucosa.

When referring to skin that is “photo damaged,” both acute photo damage and chronic photo damage is included herein. Acute photo damage can be manifest as a sunburn, and chronic photo damage can be seen as gradual changes in the skin caused by an accumulation of sun exposure throughout a period of months, but more typically years. Thus, photo damage includes human skin having experienced photo damage of some type, which can be manifest as premature aging, fine lines, wrinkles, roughness, dryness, laxity, irregular pigmentation, pre-cancerous lesions and/or skin cancers. Similarly, “skin damage” be defined to include photo damage, premature aging, fine lines, wrinkles, roughness, dryness, laxity, and/or irregular pigmentation of the skin.

The terms “drug,” “immune modulating agent,” “immune activator,” “immune modulating agent,” “immune modulator,” and “immune activating agent” can be used interchangeably and refer to a drug capable of increasing immunity of the human skin mucosa. Non-limiting examples of such drugs include imiquimod, rosiquimod, and combinations thereof. When referring generally to a “drug,” it is understood that there are various forms of a given drug, and those various forms are expressly included. In accordance with this, various drug forms include polymorphs, salts, hydrates, solvates, and cocrystals. For some drugs, one physical form of a drug may possess better physical-chemical properties making it more amenable for getting to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents. It is therefore desirable to evaluate the flux of the physical forms of a drug from non-volatile solvents to select a desirable physical form/non-volatile solvent combination.

The term “emollient,” “moisturizing agent,” “moisturizer,” and “humectant” can be used interchangeably and refer to a substance capable of softening, soothing, or enhancing a skin's ability to retain moisture in or on the surface thereof. Non-limiting examples of such compounds include glycerol, propylene glycol, dipropylenen glycol, butylene glycol, sorbitol, honey and honey derivatives such as honeyquat, urea and urea derivatives such as hydroxyethyl urea, ammonium lactate, sodium lactate, potassium lactate, pyroglutamic acid and its salts, sodium malates, polydextrose, triacetin, mannitol, oxidised polyethylene, isomalt, maltitol and maltitol syrup, lactitol, xylitol, erythrit, and combinations thereof.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced in this patent application can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic sufficient levels of the drug across, onto or into the subject's skin when the non-volatile solvent system is saturated with the drug. For systemically targeted drugs, a flux enabling non-volatile solvent system is a non-volatile solvent system that can provide therapeutically sufficient daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time.

It is also noted that once the formulation forms a solidified layer, the solidified layer can also be “flux enabling” for the drug while some of the non-volatile solvents remain in the solidified layer, even after the volatile solvents (including water) have been substantially evaporated.

Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling. The term “flux-enabling” when used with a solidified layer of the current invention refers to a solidified layer on a skin surface that is capable of generating therapeutically effective flux of the immune modulating agent. (Flux measurement tests can be done with a setup that is the same as or similar to that in Example 1).

The phrase “effective amount,” “therapeutically effective amount,” “therapeutically effective rate(s),” or the like, as it relates to a drug, refers to sufficient amounts or delivery rates of a drug which achieves any appreciable level of therapeutic results in treating a condition for which the drug is being delivered. It is understood that “appreciable level of therapeutic results” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors to some degree. However, for each drug, there is usually a consensus among those skilled in the art on the range of doses or fluxes that are sufficient in most subjects. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” or “therapeutically sufficient flux” is defined as the permeation flux of the selected drug that delivers sufficient amount of drug into or across the skin to be clinically beneficial. “Clinically beneficial” or “clinically sufficient” when referring to flux means at some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin or regional tissues or organs close to the skin surface (such as joints, certain muscles, or tissues/organs that are at least partially within 5 cm of the skin surface), “therapeutically effective flux” refers to the drug flux that can deliver a sufficient amount of the drug into the target tissues within a clinically reasonable amount of time. For drugs that target the systemic circulation, “therapeutically effective flux” refers to drug flux that, via clinically reasonable skin contact area, can deliver sufficient amounts of the selected drug to generate clinically beneficial plasma or blood drug concentrations within a clinically reasonable time. Clinically reasonable skin contact area is defined as a size of skin application area that most subjects would accept. Typically, a skin contact area of 400 cm² or less is considered reasonable. Therefore, in order to deliver 4000 mcg of a drug to the systemic circulation via a 400 cm² skin contact area over 10 hours, the flux needs to be at least 4000 mcg/400cm²/10 hour, which equals 1 mcg/cm²/hr. By this definition, different drugs have different “therapeutically effective flux.” Therapeutically sufficient flux” may be different in different subjects and or at different times for even the same subject. However, for each drug, there is usually a consensus among the skilled in the art on the range of doses or fluxes that are sufficient in most subjects at most times.

The term “plasticizing” in relation to flux-enabling non-volatile solvent(s) is defined as a flux-enabling non-volatile solvent that acts as a plasticizer for the solidifying agent. A “plasticizer” is an agent which is capable of increasing the percentage elongation of the formulation after the volatile solvent system has at least substantially evaporated. Plasticizers also have the capability to reduce the brittleness of solidified formulation by making it more flexible and/or elastic. For example, propylene glycol is a “flux-enabling, plasticizing non-volatile solvent” for the drug ketoprofen with polyvinyl alcohol as the selected solidifying agent. However, propylene glycol in a formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

The term “adhesion” or “adhesive” when referring to a solidified layer herein refers to sufficient adhesion between the solidified layer and the skin so that the layer does not fall off the skin during intended use on most subjects. Further, “adhesive” or the like when used to describe the solidified layer means the solidified layer is adhesive to the body surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the body surface. For example, excessively sweating or oily skin, or oily substances on the skin surface may make the solidified layer less adhesive to the skin. Therefore, the adhesive solidified layer of the current invention may not be able to maintain perfect contact with the body surface and deliver the drug over a sustained period of time for every subject under any conditions on the body surface. A standard is that it maintains good contact with most of the body surface, e.g. 70% of the total area, over the specified period of time for most subjects under normal conditions of the body surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

“Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, water, propanol, C4-C6 hydrocarbons, butane, isobutene, pentane, hexane, acetone, chlorobutanol, ethyl acetate, fluro-chloro-hydrocarbons, turpentine, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, and combinations thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

The term “solvent vehicle” describes compositions that include both a volatile solvent system and non-volatile solvent system. The volatile solvent system is chosen so as to evaporate from the adhesive peelable formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

The term “adhesive” in relation to the solidified layer means it is adhesive to the skin on which the original formulation was applied, and not necessarily, and preferably not, adhesive on the other side to other objects.

“Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and various aliphatic resins and aromatic resins.

The terms “washable” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, and combinations thereof. In aspect of the invention the washing solvents can be selected from the group consisting of water, ethanol, isopropyl alcohol and combinations thereof. Surfactants can also be used in some embodiments.

The term “drying time” or “acceptable length of time” refer to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, the present invention is drawn to a formulation for treating photo damaged human skin, comprising an immune modulating agent, a solvent vehicle, and a solidifying agent. The solvent vehicle can comprise a volatile solvent system including at least one volatile solvent, and a non-volatile solvent system including at least one non-volatile solvent. The formulation can have a viscosity suitable for application and adhesion to a skin surface prior to evaporation of the volatile solvent system, and can be applied as a layer to the skin surface to form a solidified layer after at least partial evaporation of the volatile solvent system. The drug can continue to be topically delivered after the volatile solvent system is substantially evaporated.

In another embodiment, a method of treating photo damaged skin can comprise applying a layer of a formulation to an area of skin exhibiting aging, wrinkles, or photo damage. The formulation can comprise an immune modulating agent, a solvent vehicle, and a solidifying agent. The solvent vehicle can include a volatile solvent system including at least one volatile solvent, and a non-volatile solvent system including at least one non-volatile solvent, wherein the non-volatile solvent system is capable of facilitating topical delivery of the immune modulating agent at a therapeutically effective rate to the skin over a sustained period of time. The formulation can have a viscosity suitable for application and adhesion to the skin surface prior to evaporation of the volatile solvent system. Other steps include solidifying the formulation on the skin to form a solidified layer by at least partial evaporation of the volatile solvent system, and dermally delivering the immune modulating agent from the solidified layer to the area of skin over a sustained period of time at a therapeutically effective rate for treating photo damaged human skin.

In another embodiment, an adhesive solidifying formulation for treating photo damaged human skin can comprise an immune modulating agent, and at least one member selected from the group consisting of isostearic acid, triacetin, sorbitan monolaurate, and combinations thereof.

In another embodiment, a solidified layer for treating photo damaged human skin can comprise an immune modulating agent, a non-volatile solvent system, and a solidifying agent. The non-volatile solvent system can comprise at least one non-volatile solvent, and the system can be capable of facilitating the delivery of the immune modulating agent at a therapeutically effective rate over a sustained period of time. The solidified layer can be capable of adhering to a human skin surface for at least two hours.

In further detail, the present invention is related to a formulation comprising an immune activator, a volatile solvent system comprising at least one volatile solvent (defined as water or solvents more volatile than water), a non-volatile solvent system comprising one or more non-volatile solvent (defined as less volatile than water), and a solidifying agent. Before application to the skin, the formulation is in its initial, less-than-solid form, such as cream, gel, paste, or ointment. After applying a layer of such a formulation on the skin area to be treated, the evaporation of the volatile solvent(s), with the help from the solidifying agent, can convert the formulation layer into a soft, flexible, coherent solid layer that is optionally peelable. The non-volatile solvent system stays in the formulation for substantially the entire duration of the application and serves as vehicle solvent for delivering the drug into the skin (a fraction of the non-volatile solvent(s) may be absorbed by skin during the application). The soft, flexible, coherent solid layer is designed to adhere to the skin for a substantial duration, preferably longer than 2 hours. Optionally, an emollient and/or a moisturizing substance can be included in the formulation for providing beneficial moisturizing effect as well as soothing the skin and minimizing the possible irritation caused by the immune activator.

The formulations of the present invention provide several advantages over the Aldara cream. First, the immune activating agent in the formulations of the present invention is “anchored” on the skin surface to be treated, and thus is not as susceptible to unintentional removal. The optional emollient and/or moisturizing agent(s) can moisturize and sooth the skin to provide additional treatment benefits. The optional emollient and/or moisturizing agent(s) may also offset the possible irritation caused by the immune activating agent. Furthermore, the physical barrier provided by the solidified layer and water retention property of the moisturizing agent can help maintain moisture in and/or on the surface of the skin. This is believed to be valuable because hydration of the skin is believed to increase the permeability of the skin, which in turn can increase the absorption of the immune activating agent. Therefore, formulations containing both immune activator and moisturizing agent can be synergistic.

These embodiments exemplify the present invention which is related to novel formulations, methods, and solidified layers that involve formulations typically in the initial form of semi-solids (including creams, gels, pastes, ointments, and other viscous liquids), which can be easily applied onto the skin as a layer, and can quickly (from 15 seconds to about 4 minutes under standard skin and ambient conditions) to moderately quickly (from about 4 to about 15 minutes under standard skin and ambient conditions) change into a solidified layer, e.g., a coherent and soft solid layer, for drug delivery. A solidified layer thus formed is capable of delivering drug to the skin, into the skin, across the skin, etc., at substantially constant rates, over an sustained period of time, e.g., hours to tens of hours, so that most of the active drug is delivered after the solidified layer is formed. In one embodiment, the formulations of the present invention can be formulated such that the solidified layer can deliver the immune modulating agent at a therapeutically effective rate over at least about 6 hours. In another embodiment, the formulations of the present invention can be formulated such that the solidified layer is capable of delivering the immune modulating agent at a therapeutically effective rate over at least about 24 hours.

Additionally, the solidified layer typically adheres to the skin, but has a solidified, minimally-adhering, outer surface which is formed relatively soon after application and which does not substantially transfer to or otherwise soil clothing or other objects that a subject is wearing or that the solidified layer may inadvertently contact. The solidified layer can also be formulated such that it is highly flexible and stretchable, and thus capable of maintaining good contact with a skin surface, even if the skin is stretched during body movement In selecting the various components that can be used, e.g., drug, solvent vehicle of volatile solvent system and non-volatile solvent system, solidifying agent(s), etc., various considerations can occur. For example, the volatile solvent system can be selected from pharmaceutically or cosmetically acceptable solvents known in the art. In one embodiment of the present invention, the volatile solvent system can include ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone and combinations thereof. In another embodiment of the present invention, the volatile solvent system can include iso-amyl acetate, denatured alcohol, methanol, propanol, isobutene, pentane, hexane, chlorobutanol, turpentine, methyl ethyl ketone, and combinations thereof. The volatile solvent system can include a mixture or combination of any of the volatile solvents set forth in the embodiments above.

Additionally, these volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 10 wt % to about 85 wt %, and more preferably from about 20 wt % to about 50 wt %.

The volatile solvent system can also be chosen to be compatible with the non-volatile solvent, solidifying agent, drug, and any other excipients that may be present. For example, polyvinyl alcohol (PVA) is not soluble in ethanol. Therefore, a volatile solvent which will dissolve PVA should to be formulated in the solidified layer in this embodiment. For instance, water will dissolve PVA and can be utilized as a volatile solvent in a solidifying formulation; however, the drying time in such a formulation may be too long to certain applications. Therefore, a second volatile solvent (e.g., ethanol) can be formulated into the solidified layer to reduce the water content but maintain a sufficient amount of water to keep PVA in solution and thereby reduce the drying time for the solidified layer.

The non-volatile solvent system can also be chosen or formulated to be compatible with the solidifying agent, the drug, the volatile solvent, and any other ingredients that may be present. For example, the solidifying agent can be chosen so that it is dispersible or soluble in the non-volatile solvent system. Most non-volatile solvent systems and solvent vehicles as a whole will be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA, a solidifying agent. In order to dissolve sufficient amount of an active drug and use PVA as a solidifying agent at the same time, a non-solvent system including PEG 400 and glycerol (compatible with PVA) in an appropriate ratio can be formulated, achieving a compatibility compromise. As a further example of compatibility, non-volatile solvent/solidifying agent incompatibility is observed when Span 20 is formulated into a solidifying formulation containing PVA. With this combination, Span 20 can separate out of the formulation and form an oily layer on the surface of the solidified layer. Thus, appropriate solidifying agent/non-volatile solvent selections are desirable in developing a viable formulation, and combinations.

In further detail, non-volatile solvent(s) that can be used alone or in combination to form non-volatile solvent systems can be selected from a variety of pharmaceutically acceptable liquids. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof. In another embodiment the non-volatile solvent system can include benzoic acid, butyl alcohol, dibutyl sebecate, diglycerides, dipropylene glycol, eugenol, fatty acids such, isopropyl myristate, mineral oil, oleyl alcohol, vitamin E, triglycerides, sorbitan fatty acid surfactants, triethyl citrate, and combinations thereof. In a further embodiment, the non-volatile solvent system can include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In a further embodiment, the non-volatile solvent system can include a combination or mixture of any of the non-volatile solvents set forth in the any of the above discussed embodiments. For example, in one embodiment, the non-volatile solvent system can include sorbitan monolaurate, isostearic acid, triacetin, and/or benzoic acid.

In addition to these and other considerations, the non-volatile solvent system can also serve as plasticizer in the adhesive formulation so that when the solidified layer is formed, the layer is flexible, stretchable, and/or otherwise “skin friendly.”

Certain volatile and/or nonvolatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the volatile solvent is capable of irritating the skin, it would be helpful to use a non-volatile solvent that is capable of reducing skin irritation. Irriation reduction solvents or compounds can also be useful for reducing the irritation caused by the delivery of certain types of drugs. Due to the mechanism of action of the immune modulators, skin areas treated by immune modulator formulations often show signs of irritation such as redness. Substances known to reduce skin irritation can be added to the formulation of the current invention to minimize the irritation, or at least minimize the signs of irritation. Examples of such substances capable of preventing or reducing skin irritation include, but are not limited to, glycerin, honey, and/or propylene glycol.

The formulations of the present invention may further comprise a pH modifying agent for adjusting the pH of the formulation to a point or a range most suitable for the delivery of the drug. This feature can be important for a drug that is ionizable.

The formulations of the current invention may also contain two or more non-volatile solvents that independently are not enabling non-volatile solvents for a drug but when formulated together become an enabling non-volatile solvent. One possible reason for these initially non enabling non-volatile solvents to become enabling non-volatile solvents when formulated together may be due to the optimization of the ionization state of the drug to a physical form which has higher flux or the non-volatile solvents act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in an adequate non-volatile solvent system include but are not limited to isostearic acid/trolamine, isostearic acid/diisopropyl amine, oleic acid/trolamine, and/or propylene glycol/isostearic acid.

The selection of the solidifying agent can also be carried out in consideration of the other components present in the adhesive formulation. The solidifying agent can be selected or formulated to be compatible to the drug and the solvent vehicle (including the volatile solvent(s) and the non-volatile solvent system), as well as to provide desired physical properties to the solidified layer once it is formed. Depending on the drug, solvent vehicle, and/or other components that may be present, the solidifying agent can be selected from a variety of agents. In one embodiment, the solidifying agent can include polyvinyl alcohol with a MW range 20,000-70,000 (Amresco), esters of polyvinylmethylether/maleic anhydride copolymer (ISP Gantrez ES-425 and Gantrez ES-225) with MW 80,000-160,000, neutral copolymer of butyl methacrylate and methyl methacrylate (degussa Plastoid B) with MW range of 120,000-180,000, dimethylaminoethyl methacrylate-butyl methacrylate-methyl methacrylate copolymer (degussa Eudragit E100) MW 100,000-200,000, ethyl acrylate-methyl methacrylate-trimethylammonioethyl methacrylate chloride copolymer MW greater than 5,000 or similar MW to Eudragit RLPO (degussa), Zein (prolamine), MW greater than 5,000 (zein, MW around 35,000, Freeman industries), pregelatinized starch having a MW similar to Instant Pure-Cote B793 (Grain Processing Corporation), ethyl cellulose MW greater than 5,000 or MW similar to Aqualon EC N7, N10, N14, N22, N50, or N100 (Hercules), fish gelatin having a MW of 20,000-250,000 (Norland Products), gelatin, other animal sources with MW greater than 5,000, acrylates/octylacrylamide copolymer with a MW greater than 5,000 or MW similar to National Starch and Chemical Dermacryl 79, or the like.

In another embodiment, the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof. In a further embodiment, the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers such as BASF's Kollicoat polymers, methacrylic acid and methacrylate based polymers such as poly(methacrylic acid), or combinations thereof. In yet another embodiment, the solidifying agent can include a combination of solidifying agents set forth in the any of the above discussed embodiments. Other polymers may also be suitable as the solidifying agent, depending on the solvent vehicle components, the drug, and the specific functional requirements of the given formulation. Other solidfying agents may also be suitable as the solidifying agent, depending on the solvent vehicle components, the drug, and the specific functional requirements of the given formulation.

In one embodiment, the non-volatile solvent system and the solidifying agent(s) should be compatible with each other. Compatibility can be defined as i) the solidifying agent does not substantially negatively influence the function of the non-volatile solvent system, except for some reduction of flux; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent(s) can be from about 0.1:1 to about 10:1. In another aspect, the ratio between the non-volatile solvent system and the solidifying agent can be from about 0.5:1 to about 2:1.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface of the solidified layer. If the drug is very potent and the solidified layer has very high tensile strength, a layer as thin as 0.01 mm may be sufficient. If the drug has rather low potency and the solidified layer has low tensile strength, a layer as thick as 2-3 mm may be desirable. Thus, for most drugs and formulations, the appropriate thickness can be from about 0.05 mm to about 3 mm, 0.1 to about 2 mm, or from about 0.05 mm to about 0.2 mm to about 0.4 mm. In one embodiment, the formulations of the present invention can have sufficient gas volatile solvents such that the formulation can be contained in a pressurized container and applied to the skin by spraying. In another embodiment the formulation can be sprayed on an affected skin surface in a layer having a thickness of about 1 to 2 mm and having an area wider than the affected skin area. In yet a further embodiment, the formulations of the present invention can be formulated to include compounds having sunscreen characteristics. In another embodiment, the formulations can be concurrently applied with a sunscreen, either within the formulation, or applied separately.

The flexibility and stretchability of a solidified layer can be desirable. As a further note, it is a unique feature of the solidified layers of the present invention that they can keep a substantial amount of the non-volatile solvent system, which can be optimized for delivering the drug on the body surface. This feature can provide unique advantages over existing products. For example, in some semi-solid formulations, upon application to a skin surface, the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

A further feature of a formulation prepared in accordance with embodiments of the present invention is related to drying time. If a formulation dries too quickly, the user may not have sufficient time to spread the formulation into a thin layer on the skin surface before the formulation is solidified, leading to poor skin contact. If the formulation dries too slowly, the subject may have to wait a long time before resuming normal activities (e.g. putting clothing on or retiring to sleep) that may remove un-solidified formulation. Thus, it can be desirable for the drying time to be longer than about 15 seconds but shorter than about 15 minutes, and preferably from about 0.5 minutes to about 4 minutes.

One way for conveniently using the formulations of the present invention is to apply the formulation on the skin to be treated within an hour of sleeping and remove the solidified layer within an hour after waking. Another way is to apply the formulation within an hour after waking and remove the solidified layer within an hour of sleeping.

Applying the formulation to cover a skin area slightly beyond the damaged skin area is believed to be potentially advantageous. Therefore, one embodiment of the current invention is to apply the formulation at least 1-2 mm beyond the damaged skin area.

Other benefits of the solidified layers of the present invention include the presence of a physical barrier that can be formed by the material itself. Another feature of the formulations of the present invention is related to solidifying formulations comprising a drug, a non-volatile solvent system comprising at least one non-volatile solvent, a solidifying agent, and a volatile solvent system comprising a volatile solvent whose boiling point is below 20° C. (such a solvent can be used as a propellant or can be dissolved in the formulation). In one embodiment, the formulation can be stored in a pressurized container and be sprayed on the skin surface with the help of the propellant. Some hydrofluorocarbons commonly used as propellants in pharmaceutical or dosmetic industries can work in this design. More specifically, the propellants may include, but not limited to dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof. The formulation may also be expelled out of the container and/or applied on the skin via a manual pump. Further, formulations comprising a room temperature gaseous volatile solvents are expected to dry much faster. Spraying the formulation onto the skin can avoid touching the skin with an applicator which can cause severe pain in the sometimes hypersensitive skin.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Hairless mouse skin (HMS) or human epidermal membrane (HEM) is used as the model membranes as noted for the in vitro flux studies described in herein. Hairless mouse skin (HMS) is used as the model membrane for the in vitro flux studies described in herein. Freshly separated epidermis removed from the abdomen of a hairless mouse is mounted carefully between the donor and receiver chambers of a Franz diffusion cell. The receiver chamber is filled with pH 7.4 phosphate buffered saline (PBS). The experiment is initiated by placing test formulations (of Examples 2-5) on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin can be used as the model membrane for the in vitro flux studies as well. The mounting of the skin and the sampling techniques used as the same as described above for the HMS studies.

### Examples 2-5

Adhesive solidifying formulations containing the following components are made:

TABLE 1
Imiquimod peelable formulation ingredients

Example

| Ingredients* | 2 | 3 | 4 | 5 |
|---|---|---|---|---|
| PVA | 10.1 | | | |
| Plastoid B** | | 17.5 | | |
| Eudragit RL PO | | | 16.2 | 24.8 |
| Pemulen TR-2 | 0.3 | | | |
| Water | 52.9 | | | |
| Isopropanol | | 35.1 | | |
| Ethanol | | | 32.4 | 38.6 |
| ISA (Isostearic Acid) | 16.8 | 23.4 | 23.1 | 27.6 |
| Salicylic Acid | 15.2 | 16.4 | 16.2 | |
| Trolamine | 1.7 | | | |
| Triacetin | | 3.5 | 3.5 | 4.1 |
| Imiquimod | 3.0 | 4.1 | 4.0 | 4.8 |

*Ingredients are noted as weight percent.
**Polymer from Degussa

These formulations are applied to HMS skin as described in Example 1, and the imiquimod flux is measured. A summary of the results from in vitro flux studies carried out with the formulations in Examples 2-5 are listed in Table 2.

TABLE 2
Steady-state flux of Imiquimod through hairless mouse skin
from various adhesive peelable formulations at 35° C.

| | Average flux | Ratio to |
|---|---|---|
| Formulation | mcg/cm²/h* | Control** |
| Example 2 | 1 ± 1 | 1.1 |
| Example 3 | 4.5 ± 0.4 | 5 |
| Example 4 | 3.8 ± 0.5 | 4.2 |
| Example 5 | 0.8 ± 0.2 | 0.9 |
| Aldara | 0.9 ± 0.02 | 1 |

*The flux values represent the mean and SD of three determinations
**Ratio to control calculated by dividing the flux value for each Example by the flux value for Aldara control flux.

In vitro flux of Examples 2-5 is substantially higher than that of the Aldara control.

### Example 6

To demonstrate the ability of the solidifying formulations to reduce the transepidermal water loss (TEWL) the following experiment was conducted.

Placebo PVA formulation was applied to the top of the hand and the TEWL was measured on a site immediately adjacent to the solidified layer and on top of the solidified layer. The TEWL measurement of the site covered by the layer was 33% lower than the untreated skin site.

Placebo Plastoid B formulation was applied to the top of the hand and the TEWL was measured on a side immediately adjacent to the solidified layer and on top of the solidified layer. The TEWL measurement on the site covered by the layer was 30% lower than the untreated skin site.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A formulation for treating photo damaged human skin, comprising:
a) an immune modulating agent;
b) a solvent vehicle, comprising:
i) a volatile solvent system comprising at least one volatile solvent, and
ii) a non-volatile solvent system comprising at least one non-volatile solvent; and
c) a solidifying agent,
wherein the formulation has a viscosity suitable for application and adhesion to a skin surface prior to evaporation of the volatile solvent system, wherein the formulation applied as a layer to the skin surface forms a solidified layer after at least partial evaporation of the volatile solvent system, and wherein the drug continues to be topically delivered after the volatile solvent system is substantially evaporated.

2. A formulation as in claim 1, which comprises a moisturizing agent.

3. A formulation as in claim 3, wherein the moisturizing agent includes at least one member selected from the group consisting of: glycerol, propylene glycol, dipropylenen glycol, butylene glycol, sorbitol, honey and honey derivatives such as honeyquat, urea and urea derivatives such as hydroxyethyl urea, ammonium lactate, sodium lactate, potassium lactate, pyroglutamic acid and its salts, sodium malates, polydextrose, triacetin, mannitol, oxidised polyethylene, isomalt, maltitol and maltitol syrup, lactitol, xylitol, erythrit, and combinations thereof.

4. A formulation as in claim 1, wherein the non-volatile solvent system acts as a plasticizer for the solidifying agent.

5. A formulation as in claim 1, wherein the volatile solvent system comprises water.

6. A formulation as in claim 1, wherein the volatile solvent system includes at least one member selected from the group of ethanol, isopropyl alcohol, and combinations thereof.

7. A formulation as in claim 1, wherein the volatile solvent system includes at least one solvent more volatile than water, and includes a member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, and combinations thereof.

8. A formulation as in claim 1, wherein the volatile solvent system includes at least one solvent more volatile than water, and includes a member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

9. A formulation as in claim 1, wherein the volatile solvent system comprises at least two volatile solvents.

10. A formulation as in claim 9, wherein the volatile solvent system comprises at least one liquid volatile solvent and at least one gaseous volatile solvent having a boiling point below 20° C.

11. A formulation as in claim 1, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

12. A formulation as in claim 1, wherein the non-volatile solvent system includes at least one member selected from the group consisting of benzoic acid, butyl alcohol, dibutyl sebecate, diglycerides, dipropylene glycol, eugenol, fatty acids, isopropyl myristate, mineral oil, oleyl alcohol, vitamin E, triglycerides, sorbitan fatty acid surfactants, triethyl citrate, and combinations thereof.

13. A formulation as in claim 1, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyidodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

14. A formulation as in claim 1, wherein the non-volatile solvent system comprises isostearic acid.

15. A formulation as in claim 1, wherein the non-volatile solvent system comprises at least one member selected from the group consisting of sorbitan monolaurate, isostearic acid, triacetin, benzoic acid, and combinations thereof.

16. A formulation as in claim 1, wherein the non-volatile solvent system comprises at least two non-volatile solvents.

17. A formulation as in claim 1, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

18. A formulation as in claim 1, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, and combinations thereof.

19. A formulation as in claim 1, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

20. A formulation as in claim 1, wherein the immune modulating agent includes multiple immune modulating agents.

21. A formulation as in claim 1, wherein the immune modulating agent includes imiquimod.

22. A formulation as in claim 1, wherein the immune modulating agent includes rosiquimod.

23. A formulation as in claim 1, wherein the formulation is formulated to deliver the drug at a therapeutically effective rate for at least about 2 hours following the formation of the solidified layer.

24. A formulation as in claim 1, wherein the formulation is formulated to deliver the drug at a therapeutically effective rate for at least about 4 hours following the formation of the solidified layer.

25. A formulation as in claim 1, wherein the formulation is formulated to deliver the drug at a therapeutically effective rate for at least about 8 hours following the formation of the solidified layer.

26. A formulation as in claim 1, wherein the formulation is formulated to deliver the drug at a therapeutically effective rate for at least about 12 hours following the formation of the solidified layer.

27. A formulation as in claim 1, wherein the weight ratio of the non-volatile solvent system to the solidifying agent is from about 0.1:1 to about 10:1.

28. A formulation as in claim 1, wherein the weight ratio of the non-volatile solvent system to the solidifying agent is from about 0.5:1 to about 2:1.

29. A formulation as in claim 1, wherein the formulation further comprises a substance capable of reducing skin irritation.

30. A formulation as in claim 30, wherein the substance capable of reducing skin irritation includes a member selected from the group consisting of glycerin, propylene glycol, honey, and combinations thereof.

31. A formulation as in claim 1, wherein the solidified layer is formed within about 15 minutes of application to the skin surface under standard skin and ambient conditions.

32. A formulation as in claim 1, wherein the solidified layer is formed within about 4 minutes of the application to the skin surface under standard skin and ambient conditions.

33. A formulation as in claim 1, wherein the formulation has an initial viscosity prior to skin application from about 100 to about 3,000,000 centipoises.

34. A formulation as in claim 1, wherein the formulation has an initial viscosity prior to skin application from about 1,000 to about 1,000,000 centipoises.

35. A formulation as in claim 1, wherein the weight percentage of the volatile solvent system is from about 10 wt % to about 85 wt %.

36. A formulation as in claim 1, wherein the weight percentage of the volatile solvent system is from about 20 wt % to about 50 wt %.

37. A formulation as in claim 1, wherein the non-volatile solvent system includes multiple non-volatile solvents, and at least one of the non-volatile solvents improves the compatibility of the non-volatile solvent system with the solidifying agent.

38. A formulation as in claim 1, wherein the solidified layer is coherent, flexible, and continuous.

39. A formulation as in claim 1, wherein the solidified layer, upon formation, is a soft, coherent solid that is peelable from a skin surface as a single piece or as only a few large pieces relative to the application size.

40. A formulation as in claim 1, wherein the solidified layer is removable from a skin surface by washing.

41. A formulation as in claim 1, wherein the volatile solvent system comprises a volatile solvent whose boiling point is below 20° C.

42. A formulation as in claim 41, wherein the volatile solvent with the boiling point below 20° C. is completely dissolved in the formulation.

43. A formulation as in claim 41, wherein the volatile solvent with the boiling point below 20° C. is included in the formulation as a propellant for pressurized spray-on application.

44. A formulation as in claim 41, wherein the volatile solvent with the boiling point below 20° C. is a hydrofluorocarbon.

45. A formulation as in claim 41, wherein the at least one solvent whose boiling point is below 20 C is selected from the group consisting of dimethyl ether, butane, 1,1, Difluoroethane, 1,1,1,2 tetrafluorethane, 1,1,1,2,3,3,3-heptafluoropropane, 1,1,1,3,3,3 hexafluoropropane, or a mixture thereof.

46. A formulation as in claim 1, wherein the solidified layer includes a surface that is adhesive to a skin surface, and an opposing surface that is not adhesive.

47. A method of treating photo damaged skin, comprising:
a) applying a layer of a formulation to an area of skin exhibiting aging, wrinkles, or photo damage, the formulation comprising:
i) an immune modulating agent,
ii) a solvent vehicle, comprising:
a volatile solvent system including at least one volatile solvent, and
a non-volatile solvent system including at least one non-volatile solvent, wherein the non-volatile solvent system is capable of facilitating topical delivery of the immune modulating agent at a therapeutically effective rate to the skin over a sustained period of time, and
iii) a solidifying agent,
wherein the formulation has a viscosity suitable for application and adhesion to the skin surface prior to evaporation of the volatile solvent system;
b) solidifying the formulation on the skin to form a solidified layer by at least partial evaporation of the volatile solvent system; and
c) dermally delivering the immune modulating agent from the solidified layer to the area of skin over a sustained period of time at a therapeutically effective rate for treating photo damaged human skin.

48. A method as in claim 47, wherein the thickness of the formulation applied on the skin is between about 0.05 mm to about 3 mm.

49. A method as in claim 47, wherein the thickness of the formulation applied on the skin is between about 0.1 mm to about 2 mm.

50. A method as in claim 47, wherein the thickness of the formulation applied on the skin is between about 0.2 mm to about 0.4 mm.

51. A method as in claim 47, wherein the formulation is applied on the skin of the subject within an hour of sleeping and removed within an hour after waking.

52. A method as in claim 47, wherein the formulation is applied on the skin of the subject after waking and removed before sleeping.

53. A method as in claim 47, wherein the solidified layer is left on the skin for at least about 2 hours.

54. A method as in claim 47, wherein the solidified layer is left on the skin for at least about 6 hours.

55. A method as in claim 47, wherein the formulation includes a moisturizing agent.

56. A method as in claim 55, wherein the moisturizing agent includes at least one member selected from the group consisting of glycerol, propylene glycol, dipropylenen glycol, butylene glycol, sorbitol, honey and honey derivatives such as honeyquat, urea and urea derivatives such as hydroxyethyl urea, ammonium lactate, sodium lactate, potassium lactate, pyroglutamic acid and its salts, sodium malates, polydextrose, triacetin, mannitol, oxidised polyethylene, isomalt, maltitol and maltitol syrup, lactitol, xylitol, erythrit, and combinations thereof.

57. A method as in claim 47, wherein the volatile solvent system includes at least one member selected from the group consisting of ethanol, isopropyl alcohol, water, dimethyl ether, diethyl ether, butane, propane, isobutene, 1,1, difluoroethane, 1,1,1,2 tetrafluorethane,1,1,1,2,3,3,3-heptafluoropropane, 1,1,1,3,3,3 hexafluoropropane, ethyl acetate, acetone, and combinations thereof.

58. A method as in claim 47, wherein the volatile solvent system includes at least one member selected from the group consisting of iso-amyl acetate, denatured alcohol, methanol, propanol, isobutene, pentane, hexane, chlorobutanol, turpentine, cytopentasiloxane, cyclomethicone, methyl ethyl ketone, and combinations thereof.

59. A method as in claim 47, wherein the volatile solvent system includes at least two volatile solvents.

60. A method as in claim 59, wherein the volatile solvent system includes at least one liquid volatile solvent and at least one gaseous volatile solvent having a boiling point less than 20° C.

61. A method as in claim 47, wherein the volatile solvent system includes at least one gaseous volatile solvent having a boiling point less than 20° C.

62. A method as in claim 60, wherein the gaseous volatile solvent includes at least one solvent selected from the group consisting of ether, dimethyl ether, propane, isobutane, difluoroethane, butane; 1,1,1,2 tetrafluorethane; 1,1,1,2,3,3,3-heptafluoropropane; and 1,1,1,3,3,3 hexafluoropropane, and combinations thereof.

63. A method as in claim 60, wherein the formulation has sufficient gaseous volatile solvent and is contained in a pressurized container so that it can be sprayed on the skin.

64. A method as in claim 47, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

65. A method as in claim 47, wherein the non-volatile solvent system includes at least one member selected from the group consisting of benzoic acid, butyl alcohol, dibutyl sebecate, diglycerides, dipropylene glycol, eugenol, fatty acids such, isopropyl myristate, mineral oil, oleyl alcohol, vitamin E, triglycerides, sorbitan fatty acid surfactants, triethyl citrate, and combinations thereof.

66. A method as in claim 47, wherein the non-volatile solvent system comprises isostearic acid.

67. A method as in claim 47, wherein the non-volatile solvent system comprises at least one solvent selected from the group consisting of sorbitan monolaurate isostearic acid, triacetin, benzoic acid, and combinations thereof.

68. A method as in claim 47, wherein the non-volatile solvent system comprises at least two non-volatile solvents.

69. A method as in claim 47 wherein the formulation is applied at least 1-2 mm beyond the damaged skin area.

70. A method as in claim 47 wherein the formulation is applied in combination with a sunscreen, either applied separately or included within the formulation.

71. A method as in claim 47, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine (Zein), pregelatinized starch, ethyl cellulose, fish gelatin, gelatin, acrylates/octylacrylamide copolymers, and combinations thereof.

72. A method as in claim 47, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, and combinations thereof.

73. A method as in claim 47, wherein the solidifying agent includes at least one member selected from the group consisting of ammonia methacrylate, carrageenan, cellulose acetate phthalate aqueous, carboxy polymethylene, cellulose acetate, cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-l-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

74. A method as in claim 47, wherein the immune modulating agent includes multiple immune modulating agents.

75. A method as in claim 47, wherein the immune modulating agent includes imiquimod.

76. A method as in claim 47, wherein the immune modulating agent includes rosiquimod.

77. A method as in claim 47, wherein the formulation is formulated to deliver the drug at a therapeutically effective rate for about 2 hours to 12 hours following the formation of the solidified layer.

78. A method as in claim 47, wherein the formulation is formulated to deliver the drug at a therapeutically effective rate for at least about 12 hours following the formation of the solidified layer.

79. A method as in claim 47, wherein the weight ratio of the non-volatile solvent system to the solidifying agent is from about 0.5:1 to about 2:1.

80. A method as in claim 47, wherein the solidified layer is formed within about 15 minutes of application to the skin surface under standard skin and ambient conditions.

81. A method as in claim 47, wherein the formulation has an initial viscosity prior to skin application from about 100 to about 3,000,000 centipoises.

82. A method as in claim 47, wherein the weight percentage of the volatile solvent system is from about 10 wt % to about 85 wt %.

83. A method as in claim 47, wherein the non-volatile solvent system includes multiple non-volatile solvents, and at least one of the non-volatile solvents improves the compatibility of the non-volatile solvent system with the solidifying agent.

84. An adhesive solidifying formulation for treating photo damaged human skin, comprising an immune modulating agent, and a member selected from the group consisting of isostearic acid, triacetin, sorbitan monolaurate, and combinations thereof.

85. A formulation as in claim 83, wherein the formulation has a viscosity suitable for application and adhesion to a skin surface prior to evaporation of the volatile solvent system, wherein the formulation applied to the skin surface as a layer forms a solidified layer after at least partial evaporation of the volatile solvent system, and wherein the drug continues to be dermally delivered after the volatile solvent system is substantially evaporated.

86. A solidified layer for treating photo damaged human skin, comprising:
a) an immune modulating agent;
b) a non-volatile solvent system capable of facilitating the delivery of the immune modulating agent at a therapeutically effective rate for a sustained period of time; and
c) a solidifying agent,
wherein the solidified layer is capable of adhering to a human skin surface for at least two hours.

87. A solidified layer as in claim 85, wherein the immune modulating agent is imiquimod.

88. A solidified layer as in claim 85, wherein solidified layer comprises isostearic acid.

89. A solidified layer as in claim 85, wherein at least one non-volatile solvent in the non-volatile solvent system acts as a plasticizer for the solidifying agent.

90. A solidified layer as in claim 85, wherein the weight ratio of the non-volatile solvent system to the solidifying agent is from about 0.5:1 to about 2:1.

91. A solidified layer as in claim 85, wherein the solidified layer is formed within 15 minutes of the application to the skin surface under standard ambient conditions

92. A solidified layer as in claim 85, wherein the solidified layer has a thickness from about 0.01 mm to about 3 mm.

93. A solidified layer as in claim 85, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

94. A solidified layer as in claim 85, wherein the solidified layer is formulated to deliver the immune modulating agent at a therapeutically effective rate for at least about 2 hours.

95. A solidified layer as in claim 85, wherein the solidified layer is formulated to deliver the immune modulating agent at a therapeutically effective rate for from 2 to 12 hours.

96. A solidified layer as in claim 85, wherein the formulation is formulated to deliver the immune modulating agent at a therapeutically effective rate for at least about 12 hours.

97. A solidified layer as in claim 85, wherein the solidified layer is a soft, coherent solid that is peelable from a skin surface as a single piece or as only a few large pieces relative to the application size.

98. A solidified layer as in claim 85, wherein the solidified layer is at least substantially devoid of volatile solvents, including water and any solvent less volatile than water, and further, dermally delivers the immune modulating agent therefrom in the at least substantial absence of the volatile solvents.

99. A solidified layer as in claim 85, wherein the solidified layer can be removed by washing.

100. A solidified layer as in claim 85, wherein the solidified layer is flux-enabling for the drug.

101. A solidified layer as in claim 85, wherein the solidified layer is adhesive to the skin surface on a first major surface surface, and is non-adhesive on an opposing major surface.

102. A solidified layer as in claim 85, wherein the solidified layer is formulated to deliver a majority the drug that is dermally deliverable therefrom while the solidified layer is substantially devoid of water and any solvent more volatile than water.
